(19) United States
(12) Patent Application Publication
(10) Pub. No.: US 2019/0385729 A1
(43) Pub. Date: Dec. 19, 2019

(54) Methods And Systems For Providing Therapeutic Guidelines To A Person Having Diabetes

(71) Applicant: Roche Diagnostics Care, Inc., Indianapolis, IN (US)

(72) Inventors: Robin Wagner, Indianapolis, IN (US); Stefan Weinert, Pendleton, IN (US); Remmert Laan, Zionsville, IN (US); Kurt Waechter, Indianapolis, IN (US); Christen Rees, Indianapolis, IN (US)

(73) Assignee: Roche Diagnostics Care, Inc., Indianapolis, IN (US)

(21) Appl. No.: 16/558,648

(22) Filed: Sep. 3, 2019

Related U.S. Application Data
(63) Continuation of application No. 12/710,430, filed on Feb. 23, 2010, now abandoned.

(51) Int. Cl. G16H 20/17; G16H 10/40

(52) U.S. Cl. CPC G16H 20/17; G16H 10/40

(57) ABSTRACT

A method is disclosed for providing therapeutic guidelines to a person having diabetes. The method comprises measuring a blood glucose (bG) level of the person for two or more days, wherein at least one bG measurement is taken per day, and the at least one daily bG measurement corresponds to one or more daily events for the person; recording the measured bG levels in a computing device; determining, by the computing device, whether the recorded bG levels are below, within, or above one or more predetermined bG ranges; an automatically providing, by the computing device, therapeutic guidelines to the person, based on whether the recorded bG levels are below, within, or above the one or more predetermined bG ranges.

### CROSS-REFERENCE TO RELATED APPLICATIONS

The present specification is a continuation of U.S. patent application Ser. No. 12/710,430, filed Feb. 23, 2010, the entirety of which is incorporated by reference herein.

### TECHNICAL FIELD

The present invention generally relates to methods and systems for providing therapeutic guidelines to a person having diabetes.

### BACKGROUND

As background, people may suffer from either Type I or Type II diabetes in which the glucose level in the blood is not properly regulated by the body. Many of these people monitor their own blood glucose levels throughout the day by using blood glucose meters. For example, a person may measure his or her blood glucose level before and after each meal.

Furthermore, a health care provider may recommend a therapeutic regimen for the person having diabetes. The regimen may provide advice on eating, exercising, and so forth, and may facilitate keeping the person's blood glucose level within a desired range. Since many factors may affect the blood glucose level of a person, it may be helpful to periodically review the history of the person's blood glucose level and determine whether and how closely the blood glucose level stays within the desired range.

Accordingly, embodiments of the present disclosure provide methods and systems for determining whether a person's blood glucose level falls within the desired range and, if not, for providing therapeutic guidelines to the person, based on the measured blood glucose levels.

### SUMMARY

In one embodiment, a method for providing therapeutic guidelines to a person having diabetes comprises: measuring a blood glucose (bG) level of the person for two or more days, wherein at least one bG measurement is taken per day, and the at least one daily bG measurement corresponds to one or more daily events for the person; recording the measured bG levels in a computing device; determining, by the computing device, whether the recorded bG levels are below, within, or above one or more predetermined bG ranges; and automatically providing, by the computing device, therapeutic guidelines to the person, based on whether the recorded bG levels are below, within, or above the one or more predetermined bG ranges.

In another embodiment, a computer-readable medium having computer-executable instructions for performing a method for providing therapeutic guidelines to a person having diabetes is disclosed. The method comprises: receiving measured blood glucose (bG) levels of the person into a computing device, wherein the measured bG levels of the person are taken for two or more days such that at least one bG measurement is taken per day, and the at least one daily bG measurement corresponds to one or more daily events for the person; recording the measured bG levels in the computing device; determining, by the computing device, whether the recorded bG levels are below, within, or above one or more predetermined bG ranges; and automatically providing, by the computing device, therapeutic guidelines to the person, based on whether the recorded bG levels are below, within, or above the one or more predetermined bG ranges.

In still another embodiment, a blood glucose meter for providing therapeutic guidelines to a person having diabetes comprises a processor, a memory, a display readable by the person, and a measuring element, wherein: the measuring element is configured to measure the blood glucose (bG) level of the person for two or more days, wherein at least one bG measurement is taken per day, and the at least one daily bG measurement corresponds to one or more daily events for the person; the processor is in electrical communication with the measuring element such that the processor is configured to read the bG level of the person measured by the measuring element; the processor is in electrical communication with the memory such that the processor is configured to record the measured bG levels in the memory; the memory comprises one or more predetermined bG ranges, such that the processor is configured to read the one or more predetermined bG ranges and the recorded bG levels and determine whether the recorded bG levels are below, within, or above the one or more predetermined bG ranges; and the processor is in electrical communication with the display such that the processor is configured to transmit therapeutic guidelines to the display, wherein the therapeutic guidelines are based on whether the recorded bG levels are below, within, or above the one or more predetermined bG ranges.

### BRIEF DESCRIPTION OF THE DRAWINGS

The embodiments set forth in the drawings are illustrative and exemplary in nature and not intended to limit the inventions defined by the claims. The following detailed description of the illustrative embodiments can be understood when read in conjunction with the following drawings, where like structure is indicated with like reference numerals and in which:

FIG. 1 depicts a blood glucose meter according to one or more embodiments shown and described herein;

FIG. 2 depicts a personal computer according to one or more embodiments shown and described herein;

FIG. 3 depicts a flow diagram of one method for providing therapeutic guidelines according to one or more embodiments shown and described herein;

FIG. 4 depicts a flow diagram of one method for providing therapeutic guidelines according to one or more embodiments shown and described herein;

FIGS. 5A-B depict a flow diagram of one method for determining therapeutic guidelines according to one or more embodiments shown and described herein; and

FIG. 6 depicts therapeutic guidelines according to one or more embodiments shown and described herein.

### DETAILED DESCRIPTION

The embodiments described herein generally relate to methods and systems for providing therapeutic guidelines to people having diabetes.

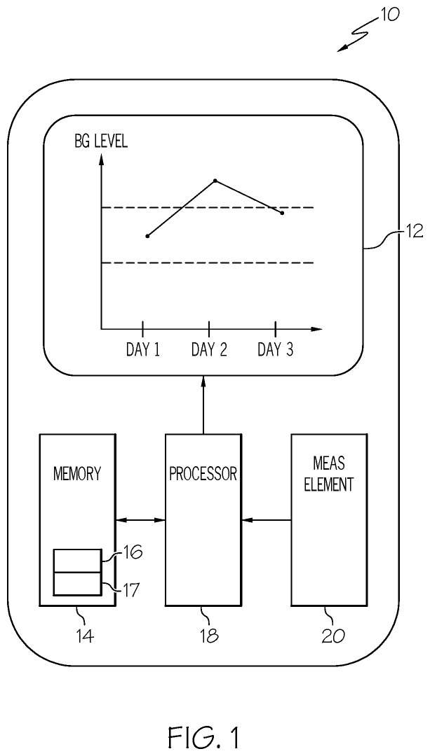

FIG. 1 depicts a blood glucose (bG) meter 10 according to one embodiment of the present disclosure. The bG meter 10 may comprise a display 12, a memory 14, a processor 18, and a measuring element 20. The measuring element 20 may be configured to measure the bG level of a person such as, for example, by using a blood sample from the person. The measuring element 20 may be in electrical communication with the processor 18 such that the processor is configured to read the bG measurement from the measuring element 20. The memory 14 may be in electrical communication with the processor 18 such that the processor 18 may record (or store) the bG measurement in the memory 14. The processor 18 may be configured to read a plurality of bG measurements per day from the measuring element 20 and may be configured to record each of these bG measurements in the memory 14. Furthermore, the processor 18 may be configured to record bG measurements from two or more days in the memory 14. For example, one month's worth of bG measurements may be recorded in the memory 14. The bG meter 10 may be configured such that it may transmit some or all of the stored bG measurements to another device, either via a wired or wireless connection (not shown).

The memory 14 may also comprise one or more predetermined bG ranges 16 for the person. As an example, a first predetermined bG range 16 may be approximately 81 to approximately 140 mg/dl (milligrams of glucose per deciliter of blood), while a second predetermined bG range may be approximately 81 to approximately 110 mg/dl. Other ranges may be used as well and may depend on the characteristics of the person. Blood glucose levels which fall below the predetermined bG range (i.e., below 81 mg/dl in the above examples) may be considered “hypoglycemic.” Similarly, blood glucose levels which fall above the predetermined bG range (i.e., above 140 mg/dl for the first range or 110 mg/dl for the second range in the above examples) may be considered “hyperglycemic.” Consequently, blood glucose levels which fall within these predetermined bG ranges may be considered “normal.” As disclosed herein, one or more predetermined bG ranges may be used, such that a first predetermined bG range may be used for some of the measured bG results, while a second predetermined bG range may be used for other measured bG results. Any number of predetermined bG ranges may be used.

The memory 14 may further comprise a blood glucose (bG) excursion amount 17. Blood glucose levels which are below the predetermined bG range 16 by at least the bG excursion amount may be considered “severe hypoglycemic.” Similarly, blood glucose levels which are above the predetermined bG range 16 by at least the bG excursion amount 17 may be considered “severe hyperglycemic.” As an example, the predetermined bG range 16 may be 81 to 140 mg/dl, and bG excursion amount may be 50 mg/dl. In this example, a bG level of 141 to 189 mg/dl may be considered hyperglycemic; and a bG level of 190 mg/dl and above may be considered severe hyperglycemic. Continuing with this example, a bG level of 31 to 80 mg/dl may be considered hypoglycemic; and a bG level of 30 mg/dl and below may be considered severe hypoglycemic. Whether the person's bG level falls below, within, or above a predetermined amount may be subsequently used by the processor 18 to provide therapeutic guidelines to the person.

The bG meter 10 may further comprise a display 12, which may be readable by the operator. The display 12 may be in electrical communication with the processor 18 such that the processor is configured to send information to the display 12. As an example, the processor 18 may send either graphical or textual information to the display 12 which may provide therapeutic guidelines to the operator. Graphical information may include X-Y graphs of the person's bG level history or other suitable information. Textual information may include text messages, such as “Your bG level is 117 mg/dl.” Both graphical and textual information may be display simultaneously, if desired. The display 12 may be a liquid crystal display (LCD) or other suitable display.

The bG meter 10 may be configured to measure the bG level of the person for two or more days. At least one bG measurement may be taken per day, and each bG measurement may correspond to at least one daily event for the person. Generally, the daily events may take place at approximately the same time each day. Daily events may include, but are not limited to, eating breakfast, eating lunch, eating dinner, and going to sleep. Other daily events may be used as well, including daily events which may affect the bG level of the person such as, but not limited to, exercising and taking medication. Regarding the three daily meals, breakfast generally may be eaten in the morning, lunch may be eaten around noon, and dinner may be eaten in the late afternoon or evening, although the meals may be eaten at other times as well, depending on the person's sleep schedule. As an example, a person working third shift (e.g., working approximately midnight to 8:00 am) may eat “dinner” at 9:00 am, may sleep from noon to 8:00 pm, may eat “breakfast” at 8:30 pm, and may eat “lunch” at 1:00 am. Other such sleep or eating schedules are contemplated as well.

The bG meter 10 may be configured to measure the person's bG level for three consecutive days, for example. For each day, the bG meter 10 may be configured to measure the person's bG level before and after each of the three daily meals (i.e., breakfast, lunch, and dinner) as well as before the person goes to sleep. In this fashion, the bG meter may be configured to take at least seven bG measurements per day. In addition, the person's bG level may be taken approximately two hours after ingesting a meal. This may provide a more accurate bG level measurement for the person.

After the bG measurements for the two or more days have been recorded in the memory 14, the processor 18 may be configured to read the bG measurements and the one or more predetermined bG ranges 16 from the memory 14. The processor 18 may then determine whether each bG measurement falls below, within, or above one of the one or more predetermined bG ranges. Based on these determinations, the processor 18 may be configured to transmit therapeutic guidelines to the display 12.

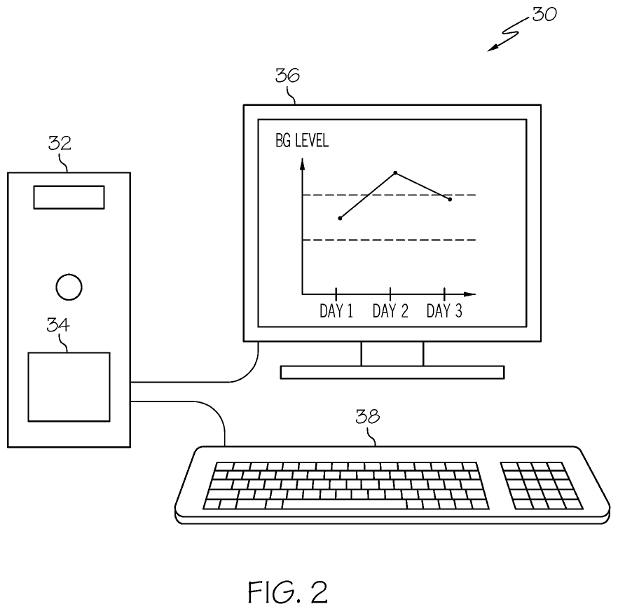

FIG. 2 illustrates another embodiment of the present disclosure in which a computing device 30 may comprise a processor 32, memory 34, a display 36, and an input device 38. The processor 32, memory 34, and display 36 may function in the same manner as the same-named elements shown in FIG. 1 and described herein. The input device 38 may comprise a keyboard, such as a “hard keyboard,” which has physical, dedicated buttons the person may press. Alternatively, input device 38 may comprise a touch screen (not shown), which permits the person to enter information by pressing certain locations on the display 36. Other types of input devices may be used as well, as is known in the art.

The computing device 30, although depicted as a desktop personal computer in FIG. 2, may also be a laptop computer, a cellular phone, a smart phone, a personal digital assistant, or any suitable device. The computing device 30 may be configured to allow the bG measurements to be manually entered into the computing device 30 via the input device 38. As an example, the person may type the bG measurements into the computing device 30 through a keyboard. Alternatively, the bG measurements may be transmitted to the computing device 30 through a wired or a wireless interface. For example, the computing device 30 may wirelessly receive bG measurements from a bG meter via a Bluetooth interface. In this fashion, the computing device 30 may automatically receive the bG measurements. Once the computing device 30 has received the bG measurements, it may record the measurements, determine whether the measurements are below, within, or above the predetermined bG range, and provide therapeutic guidelines to the person, based on whether the recorded bG levels are below, within, or above the predetermined bG range.

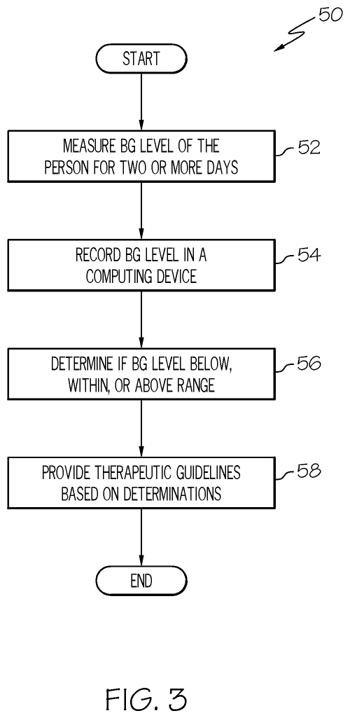

FIG. 3 depicts a flow diagram 50 of a method for providing therapeutic guidelines to a person having diabetes. This method may be performed on a bG meter, such as the one shown in FIG. 1 or any other suitable device. Act 52 of the method may measure a blood glucose (bG) level of the person for two or more days, wherein at least one bG measurement is taken per day, and the at least one daily bG measurement corresponds to at least one daily event for the person. Act 54 of the method may record the measured bG levels in a computing device. Act 56 of the method may determine whether the recorded bG levels are below, within, or above one or more predetermined bG ranges. And act 58 of the method may provide therapeutic guidelines to the person, based on whether the recorded bG levels are below, within, or above the one or more predetermined bG ranges. The acts of the method may be performed in any suitable order.

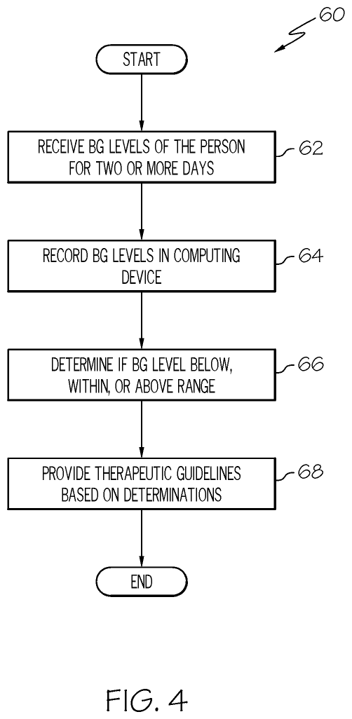

FIG. 4 depicts another flow diagram 60 of a method for providing therapeutic guidelines to a person having diabetes. This method may be stored on a computer-readable medium having computer-executable instructions for performing the method. A computer-readable medium may include, but is not limited to, a compact disc (CD), a USB thumb drive, an optical drive, or a magnetic drive. Other types of computer-readable media may be used as well, such as those presently known in the art and those yet to be discovered. The method may comprise the following acts. Act 62 of the method may receive measured blood glucose (bG) levels of the person into a computing device, wherein the measured bG levels of the person are taken for two or more days such that at least one bG measurement is taken per day, and the at least one daily bG measurement corresponds to one or more daily events for the person. Act 64 of the method may record the measured bG levels in the computing device. Act 66 of the method may determine, by the computing device, whether the recorded bG levels are below, within, or above one or more predetermined bG ranges. And act 68 of the method may automatically provide, by the computing device, therapeutic guidelines to the person, based on whether the recorded bG levels are below, within, or above the one or more predetermined bG ranges. The acts of the method may be performed in any suitable order.

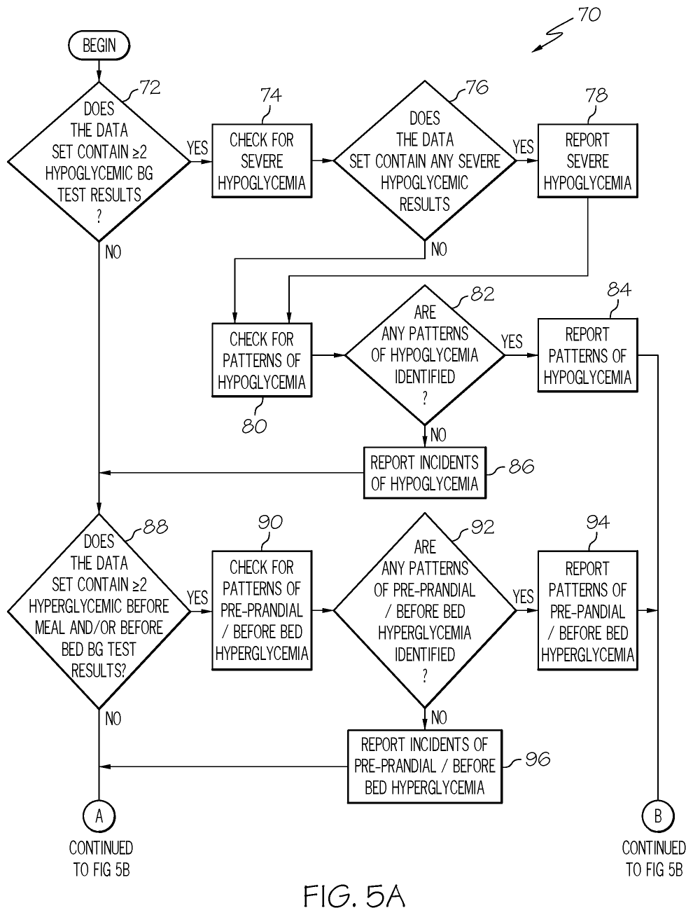

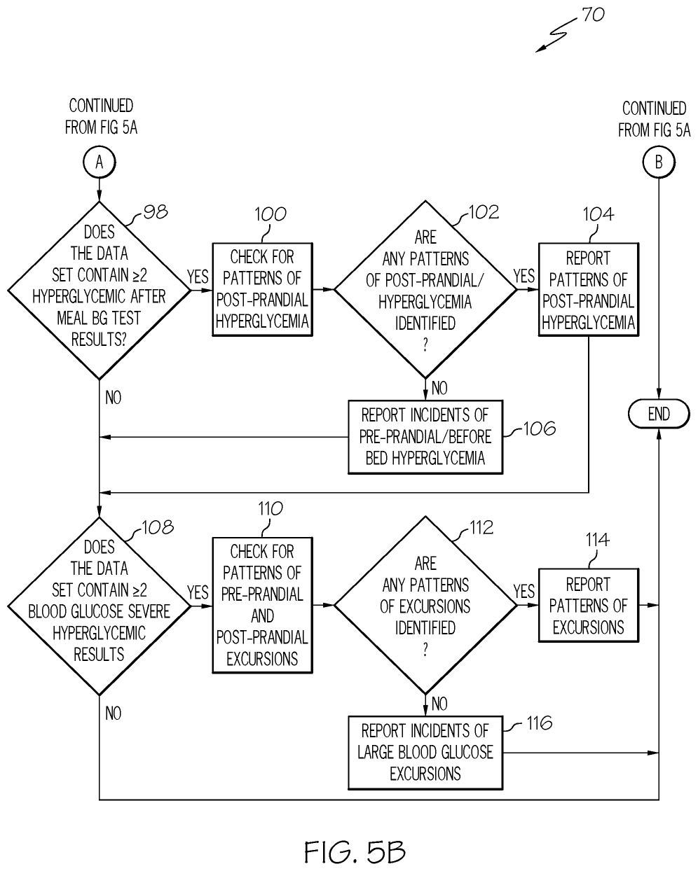

FIGS. 5A-B depict a flow diagram 70 of a method, according to one embodiment, for providing therapeutic guidelines based on whether the recorded bG levels are below, within, or above one or more predetermined bG ranges. As previously defined herein, a hypoglycemic bG level is one that is below the predetermined bG range, but by an amount that is less than the bG excursion amount; and a severe hypoglycemic bG level is one below the predetermined bG range by the bG excursion amount or more. Similarly, a hyperglycemic bG level is one that is above the predetermined bG range, but by an amount that is less than the bG excursion amount; and a severe hyperglycemic bG level is one that is above the predetermined bG range by the bG excursion amount or more.

Act 72 of the method determines whether two or more of the measured bG levels are considered hypoglycemic. If Yes, the flow diagram 70 advances to act 74; if No, the flow diagram 70 advances to act 88. At act 74, the bG results are checked for severe hypoglycemia. The flow diagram 70 then advances to act 76, where it is determined whether any bG levels are considered severe hypoglycemic. If Yes, then the severe hypoglycemic bG levels are reported to the operator at act 78. If No, the flow diagram 70 advances to act 80, wherein the bG levels are checked for patterns of hypoglycemia.

A “pattern” may occur, for example, if two or more hypoglycemic bG levels are found before or after the same daily event, such as after breakfast. As another example, a “pattern” may occur if two or more hyperglycemic bG levels are found before or after similar daily events, such as after meals (e.g., after breakfast, after lunch, and/or after dinner). Other definitions for “pattern” may be used as well.

Continuing with the flow diagram 70, act 82 of the method determines whether any hypoglycemic bG levels exhibit a pattern. If Yes, the flow diagram 70 advances to act 84, wherein the pattern (or patterns) of hypoglycemic bG levels is reported; the flow diagram 70 subsequently ends. If No, the flow diagram 70 advances to act 86, wherein the individual hypoglycemic bG levels are reported; the flow diagram 70 then advances to act 88.

At act 88, it is determined whether two or more of the bG measurement levels are considered hypoglycemic either at a pre-meal measurement or the pre-sleep (e.g., before bed) measurement. If Yes, the flow diagram 70 advances to act 90; if No, the flow diagram 70 advances to act 98. At act 90, the bG levels are checked for patterns of pre-meal or pre-sleep hyperglycemia. The flow diagram 70 then advances to act 92, wherein it is determined whether there are any patterns of pre-meal or pre-sleep hyperglycemia. If Yes, the flow diagram 70 advances to act 94, wherein the pattern (or patterns) of pre-meal or pre-sleep hypoglycemic bG levels is reported; the flow diagram 70 subsequently ends. If No, the flow diagram 70 advances to act 96, wherein the incidents of pre-meal and/or pre-sleep hypoglycemic bG levels are reported; the flow diagram 70 then advances to act 98.

Act 98 of the method determines whether two or more of the bG measurement levels are considered hyperglycemic at a post-meal (i.e., post-prandial) bG level measurement. If Yes, the flow diagram 70 advances to act 100; if No, the flow diagram 70 advances to act 108. At act 100, the bG levels are checked for patterns of post-meal hyperglycemia. The flow diagram 70 then advances to act 102, wherein it is determined whether there are any patterns of post-meal hyperglycemia. If Yes, the flow diagram 70 advances to act 104, wherein the pattern of post-meal hyperglycemic bG levels is reported; the flow diagram subsequently advances to act 108. If No, the flow diagram 70 advances to act 106, wherein the individual post-meal hyperglycemic bG levels are reported; the flow diagram 70 then advances to act 108.

At act 108, it is determined whether two or more of the bG measurement levels are considered severe hyperglycemic. If Yes, the flow diagram 70 advances to act 110; if No, the flow diagram 70 ends. At act 110, the bG levels are checked for patterns of pre-meal or post-meal severe hyperglycemia. The flow diagram 70 then advances to act 112, wherein it is determined whether there are any patterns of pre-meal or post-meal severe hyperglycemia. If Yes, the flow diagram 70 advances to act 114, wherein the pattern (or patterns) of pre-meal or post meal severe hyperglycemic bG levels are reported; the flow diagram 70 subsequently ends. If No, the flow diagram advances 70 to act 116, wherein the individual pre-meal and/or post-meal severe hyperglycemic bG levels are reported; the flow diagram 70 then ends.

The acts of the flow diagram 70 may be performed in any suitable order. Furthermore, as described herein, any number of techniques may be employed to determine whether there is a “pattern” in the bG measurement levels. For example, if bG measurements are taken for three consecutive days, a pattern may be defined as two or more hypoglycemic or hyperglycemic bG measurements before or after the same event. In this example, two pre-breakfast hypoglycemic bG measurement levels constitute a pattern. Other ways of defining a pattern may be used as well. In another example having five consecutive days of bG measurements, a pattern may be defined as five anomalous (e.g., not within the predetermined bG range) bG measurements before or after the same event. In this example, five pre-sleep hyperglycemic bG measurement levels constitute a pattern. In yet another example having three consecutive days of bG measurements, a pattern may be defined as two or more anomalous bG measurements after any meal (e.g., breakfast, lunch, or dinner). In this example, one hyperglycemic bG measurement level after breakfast on the first day, and another hyperglycemic bG measurement level after lunch on the third day constitute a pattern. Thus, it is contemplated that a definition of a “pattern” is very broad and may encompass a number of factors.

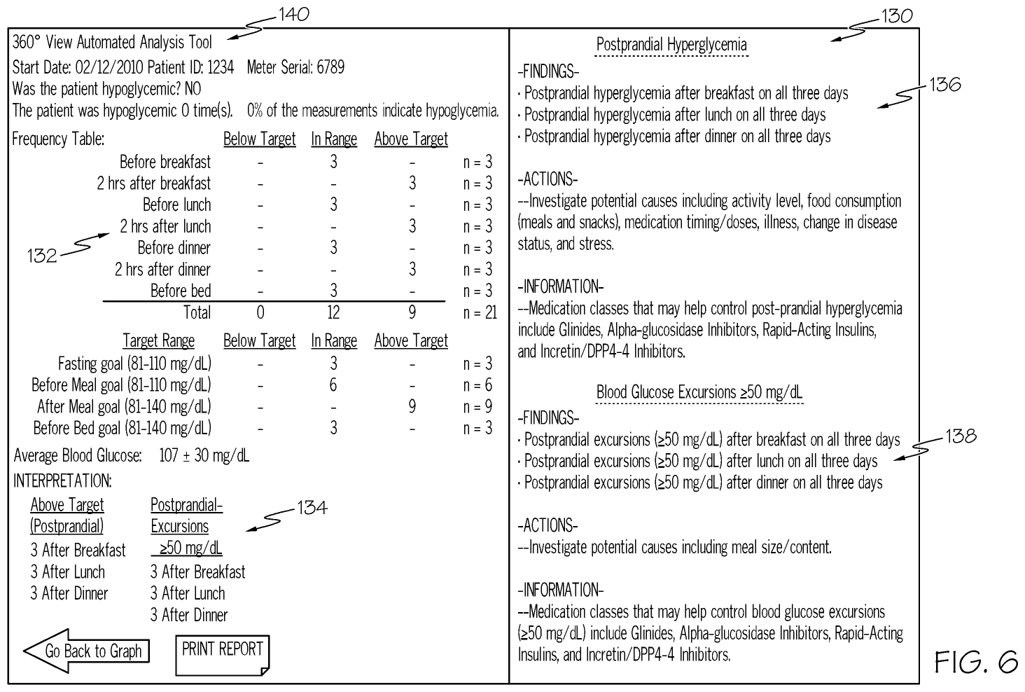

FIG. 6 depicts examples of therapeutic guidelines 130 according to one or more embodiments shown and described herein. The therapeutic guidelines 130 may be presented in graphic or textual form and may comprise a frequency table 132, a summary area 134, a hypoglycemic/hyperglycemic area 136, and a bG excursion area 138. The therapeutic guidelines 130 may further comprise an information area 140 which may provide basic information about the person and/or bG meter used.

The frequency table 132 may display the measured bG results in a tabular form and may be organized by the daily events to which each measurement corresponds. For example, the frequency table 132 may identify some or all of the following: (1) The number of hypoglycemic bG measurements for each time period, (2) The number of hyperglycemic bG measurements for each time period, (3) The number of normal bG measurements for each time period, (4) The total number of bG measurements for each time period, (5) The total number of hypoglycemic bG measurements, (6) The total number of hyperglycemic bG measurements, (7) The total number of normal bG measurements, and (8) The total number of bG measurements in the data set.

As shown in FIG. 6, there may be a row in the frequency table 132 for before breakfast bG levels, after breakfast bG levels, etc. The bottom of the frequency table 132 may indicate the one or more predetermined bG ranges (called “Target Range” in the table). As an example, the pre-meal predetermined bG range may be approximately 81 mg/dl to approximately 110 mg/dl, and the post-meal and pre-sleep predetermined bG range may be approximately 81 mg/dl to approximately 140 mg/dl. Other predetermined bG ranges may be used as well.

The rows of the frequency table 132 may be labeled to indicate the time period (e.g., before breakfast, etc.) The columns of the time period frequency table may be labeled to indicate the range determination (e.g., below, within, or above the predetermined bG range). The summary frequency table may identify the number of hypoglycemic, normal, and hyperglycemic bG measurement levels for each of the following events: pre-breakfast, pre-lunch, pre-dinner, post-breakfast, post-lunch, post-dinner, and pre-sleeping. The rows of the summary frequency table may be labeled to indicate the time periods, while the columns may be labeled to indicate the range determination. Other ways of organizing the information may be used as well.

The summary area 134 may provide a synopsis of the recorded bG levels, as shown in FIG. 6. The hypoglycemic/hyperglycemic area 136 may indicate whether there were any hypoglycemic and/or hyperglycemic bG results. The hypoglycemic/hyperglycemic area 136 may provide text indicating findings, may propose actions, and may provide additional information when one or more hypoglycemic and/or hyperglycemic bG results are found. As an example, the hypoglycemic/hyperglycemic area 136 may suggest that the person, upon finding one or more hyperglycemic bG results, “Investigate potential causes including activity level, food consumption (meals and snacks), medication timing/doses, illness, change in disease status, and stress.” The bG excursion area 138 may provide text indicating findings, may propose actions, and may provide additional information when one or more recorded bG levels are below or above the one or more predetermined bG ranges by at least the bG excursion amount (e.g., severe hypo- or hyperglycemic results). The bG excursion amount may be, for example, 50 mg/ml. In FIG. 6, as an example, the bG excursion area 138 may state, “Investigate potential causes including meal size/content. Other areas, both graphic and textual, may be included in the therapeutic guidelines 130.

The methods and systems described herein for providing therapeutic guidelines may permit the person having diabetes to modify some or all of the operating parameters on which the therapeutic guidelines may be based. Such operating parameters may include, but are not limited to, the starting and ending dates for the bG measurements, how many daily bG measurements are taken, which daily events correspond to the bG measurements, and so forth.

The methods and systems may also allow the person to enter relevant information about himself/herself and/or the bG meter, some of which may be displayed in the information area 140. As an example, the person may enter his/her name, the bG meter type, and the serial number of the bG meter, which may be stored in the memory along with the bG measurement levels. Furthermore, the person may enter information about how he/she is feeling, whether he/she is tired, etc. This latter type of information may be entered for each bG measurement, if desired, so that other patterns (other than those relating to the bG levels) may be recognized, either by the processor or by the person (e.g., upon seeing a report).

In addition to reporting incidents and patterns in the measured bG levels, the methods and systems described herein may also be configured to provide graphical information, either on a display or via a printer. For example, a graph of the person's pre-sleep bG level may be graphically shown for the two or more consecutive days (e.g., see the graph on the display 12 of FIG. 1). Alternatively, all measured bG levels may be graphically shown, such that each pre- and post-event bG levels have their own color or other identifying characteristic. As an example, all pre-breakfast bG levels may be depicted in red, all post-breakfast bG levels may be depicted in orange, etc. The graph may also highlight which bG measurements are normal, hypoglycemic, severe hypoglycemic, hyperglycemic, and/or sever hyperglycemic.

The methods and systems described herein may provide therapeutic guidelines to the person, based on whether the recorded bG levels are below, within, or above the predetermined bG range. The therapeutic guidelines may also be based on whether the recorded bG levels are considered severe hypoglycemic or severe hyperglycemic (i.e., they are below or above the predetermined bG range by at least the bG excursion amount). The following examples illustrate how the therapeutic guidelines may be determined.

If the recorded bG levels contain two or more hypoglycemic levels and the recorded bG levels contains one or more severe hypoglycemic levels, the therapeutic guidelines may report the following finding: “SEVERE HYPOGLYCEMIA.” If the recorded bG levels contain three hypoglycemic levels for the before breakfast time period, the guidelines may indicate a pattern of hypoglycemia and provide the following finding: “Pre-prandial hypoglycemia before breakfast on all three days.” If the recorded bG levels contains exactly two hypoglycemic bG test results for the before breakfast time period, the guidelines may indicate a pattern of hypoglycemia and provide one of the following findings (depending on the days when the results occurred): “Pre-prandial hypoglycemia before breakfast on days 1 and 2,” “Pre-prandial hypoglycemia before breakfast on days 2 and 3,” or “Pre-prandial hypoglycemia before breakfast on days 1 and 3.” The same may be done for bG results measured before lunch or dinner.

If the recorded bG levels contain three hypoglycemic bG test results for the after breakfast time period, the guidelines may indicate a pattern of hypoglycemia and provide the following finding: “Post-prandial hypoglycemia after breakfast on all three days.” If the recorded bG levels contains exactly two hypoglycemic bG test results for the after breakfast time period, the guidelines may indicate a pattern of hypoglycemia and provide one of the following findings (depending on the days when the results occurred): “Post-prandial hypoglycemia before breakfast on days 1 and 2,” “Post-prandial hypoglycemia before breakfast on days 2 and 3,” or “Post-prandial hypoglycemia before breakfast on days 1 and 3.” The same may be done for bG levels measured after lunch or dinner.

If the recorded bG levels contain three hypoglycemic bG test results before the sleep time period, the guidelines may indicate a pattern of hypoglycemia and provide the following finding: “Hypoglycemia before sleep on all three days.” If the recorded bG levels contains exactly two hypoglycemic bG test results for the pre-sleep time period, the guidelines may indicate a pattern of hypoglycemia and provide one of the following findings (depending on the days when the results occurred): “Hypoglycemia before sleep on days 1 and 2,” “Hypoglycemia before sleep on days 2 and 3,” or “Hypoglycemia before sleep on days 1 and 3.”

If the recorded bG levels contain two or more hypoglycemic bG test results, but no pattern of hypoglycemia is identified, the guidelines may provide the incidents of hypoglycemia and provide the following finding: “There were two or more occurrences of hypoglycemia, but no pattern was detected.” If the recorded bG levels contain two or more hypoglycemic bG test results and the recorded bG levels contain one or more severe hypoglycemic results, the guidelines may report the following guideline: “DETERMINE CAUSE IMMEDIATELY.”

If any pattern of hypoglycemia is identified, the guidelines may suggest the following actions: “1) Investigate potential causes of hypoglycemia including activity level, food consumption (meals and snacks), medication timing/doses and illness. 2) Resolve prior to addressing other blood glucose abnormalities.”

If the recorded bG levels contain two or more hypoglycemic bG test results, but no pattern of hypoglycemia is identified, the guidelines may suggest the following action: “Investigate potential causes of hypoglycemia including activity level, food consumption (meals and snacks), medication timing/doses and illness.”

If the recorded bG levels contains two or more hypoglycemic bG test results, the guidelines may provide the following information: “Medication classes that may cause hypoglycemia include: Sulfonylureas, Glinides, Long-Acting Insulins, Rapid-Acting Insulins, and various fixed dose insulin combinations.” If any pattern of hypoglycemia is identified, the guidelines may report no findings, actions, or information for hyperglycemia or severe hyperglycemia.

If no pattern of hypoglycemia is identified, and the recorded bG levels contain three hyperglycemic results for the before breakfast time period, the guidelines may indicate a pattern of pre-prandial/pre-sleep hyperglycemia and provide the following finding: “Pre-prandial hypoglycemia before breakfast on all three days.” If no pattern of hypoglycemia is identified, and the recorded bG levels contains exactly two hyperglycemic bG test results for the before breakfast time period, the guidelines may indicate a pattern of hyperglycemia and provide one of the following findings (depending on the days when the results occurred): “Pre-prandial hyperglycemia before breakfast on days 1 and 2,” “Pre-prandial hyperglycemia before breakfast on days 2 and 3,” or “Pre-prandial hyperglycemia before breakfast on days 1 and 3.” The same may be done for bG levels measured before lunch or dinner.

If no pattern of hypoglycemia is identified, and the recorded bG levels contain three hyperglycemic bG test results for the before sleep time period, the guidelines may indicate a pattern of pre-prandial/pre-sleep hyperglycemia and provide the following finding: “Hyperglycemia before sleep on all three days.” If no pattern of hypoglycemia is identified, and the recorded bG levels contains exactly two hyperglycemic bG test results for the before sleep time period, the guidelines may indicate a pattern of pre-prandial/pre-sleep hyperglycemia and provide one of the following findings (depending on the days when the results occurred): “Hyperglycemia before sleep on days 1 and 2,” “Hyperglycemia before sleep on days 2 and 3,” or “Hyperglycemia before sleep on days 1 and 3.”

If no pattern of hypoglycemia is identified, no pattern of pre-prandial/pre-sleep hyperglycemia is identified, and the recorded bG levels contain two or more before meal and/or before sleep hyperglycemic bG test results, the guidelines may provide incidents of pre-prandial /pre-sleep hyperglycemia and provide the following finding: “There were two or more occurrences of hyperglycemia, but no pattern was detected.”

If no pattern of hypoglycemia is identified, and the recorded bG levels contain two or more before meal and/or before sleep hyperglycemic bG test results, the guidelines may suggest the following actions: “1) Investigate potential causes of hyperglycemia including activity level, food consumption (meals and snacks), medication timing/doses, illness, change in disease status, and stress. 2) Resolve pre-meal and bedtime hyperglycemia before addressing post-prandial hyperglycemia.”

If no pattern of hypoglycemia is identified but the recorded bG levels contains two or more before meal and/or before sleep hyperglycemic bG levels, the guidelines may provide the following information: “1) Medication classes that may help control fasting, pre-prandial, or pre-sleep hyperglycemia include: Sulfonylureas, TZDs, Biguanides, Long-Acting Insulins, and various fixed dose insulin combinations. 2) Resolving pre-meal and bedtime hyperglycemia may reduce post-prandial hyperglycemia.” If any pattern of pre-prandial/before bed hyperglycemia is identified, the therapeutic guidelines may report not findings, actions, or information regarding post-prandial hyperglycemia or severe hyperglycemia.

If no pattern of hypoglycemia is identified, no pattern of pre-prandial/pre-sleep hyperglycemia is identified, and the recorded bG levels contain three hyperglycemic bG test results for the after breakfast time period, the guidelines may indicate a pattern of post-hyperglycemia and provide the following finding: “Post-prandial hyperglycemia after breakfast on all three days.” If no pattern of hypoglycemia is identified and no pattern of pre-prandial /pre-sleep hyperglycemia bG test results for the after breakfast time period is identified, the guidelines may indicate a pattern of post-prandial hyperglycemia and provide one of the following findings (depending on the days when the results occurred): “Post-prandial hyperglycemia after breakfast on days 1 and 2,” “Post-prandial hyperglycemia after breakfast on days 2 and 3,” or “Post-prandial hyperglycemia after breakfast on days 1 and 3.” The same may be done for bG levels measured after lunch or dinner.

If no pattern of hypoglycemia is identified, no pattern of pre-prandial/pre-sleep hyperglycemia is identified, and the recorded bG levels contain two or more after meal hyperglycemic bG test results, the guidelines may provide incidents of post-prandial hyperglycemia and provide the following finding: “There were two or more occurrences of hyperglycemia, but no pattern was detected.”

If no pattern of hypoglycemia is identified, no pattern of pre-prandial/pre-sleep hyperglycemia is identified, and the recorded bG levels contains two or more after meal hyperglycemic bG test results, the guidelines may suggest the following action: “Investigate potential causes of hyperglycemia including activity level, food consumption (meals and snacks), medication timing/doses, illness, change in disease status, and stress.”

If any pattern of post-prandial hyperglycemia is identified, the guidelines may provide the following information: “Medication classes that may help control post-prandial hyperglycemia include Glinides, Alpha-glucosidase Inhibitors, Rapid-Acting Insulins, and Incretin/DPP4-4 Inhibitors.”

If no pattern of hypoglycemia is identified, no pattern of pre-prandial/pre-sleep hyperglycemia is identified, no pattern of post-prandial hyperglycemia is identified, and the recorded bG levels contains two or more after meal hyperglycemic bG levels, the guidelines may provide the following information: “Medication classes that may help control post-prandial hyperglycemia and blood glucose excursions ≥x mg/dL include Glinides, Alpha-glucosidase Inhibitors, Rapid-Acting Insulins, and incretin/DPP4-4 Inhibitors,” where “x” is the bG excursion amount.

If no pattern of hypoglycemia is identified, no pattern of pre-prandial/pre-sleep hyperglycemia is identified, and the recorded bG levels contains three severe hyperglycemic bG levels (e.g., bG levels above the predetermined range by the bG excursion amount or more) from before breakfast to after breakfast, the guidelines may indicate a pattern of severe post-prandial excursions and provide the following finding: “Post-prandial excursions ≥x mg/dL after breakfast on all three days,” where “x” is the bG excursion amount. If no pattern of hypoglycemia is identified, no pattern of pre-prandial/pre-sleep hyperglycemia is identified, and the recorded bG levels contains exactly two severe hyperglycemic bG levels from before breakfast to after breakfast, the guidelines may indicate a pattern of large post-prandial excursions and report one of the following findings (depending on the days when the excursions occurred): “Post-prandial excursions ≥x mg/dL after breakfast on days 1 and 2,” “Post-prandial excursions ≥x mg/dL after lunch on days 2 and 3,” or “Post-prandial excursions ≥x mg/dL after lunch on days 1 and 3,” where “x” is the bG excursion amount. The same may be done for bG levels measured before and after lunch as well as before and after dinner.

If any pattern of post-prandial severe hyperglycemic bG levels is identified, the guidelines may suggest the following action: “Please investigate potential causes of post-prandial excursions ≥x mg/dL including meal size/content” and/or “Medication classes that may help control post-prandial hyperglycemia and blood glucose excursions ≥x mg/dL include Glinides, Alpha-glucosidase Inhibitors, Rapid-Acting Insulins, and incretin/DPP4-4 Inhibitors,” where “x” is the bG excursion amount.

If no pattern of hypoglycemia is identified, no pattern of pre-prandial/pre-sleep hyperglycemia is identified, no pattern of pre-prandial excursions is identified, no pattern of post-prandial severe hyperglycemia is identified, and the recorded bG levels contains two or more blood glucose excursions ≥x mg/dL, the guidelines may provide incidents of large blood glucose excursions and provide the following finding: “The patient has experienced blood glucose excursions ≥x mg/dL at least two times, but no pattern was detected,” where “x” is the bG excursion amount.

If no pattern of hypoglycemia is identified and no pattern of pre-prandial/pre-sleep hyperglycemia is identified, no pattern of pre-prandial severe hyperglycemia is identified, no pattern of post-prandial hyperglycemia is identified, and the recorded bG levels include two or more severe hyperglycemic bG levels (e.g., blood glucose excursions ≥x mg/dL, where “x” is the bG excursion amount), the guidelines may suggest the following actions: “1) Please investigate causes of post-prandial excursions including meal size/content. 2) Please investigate potential causes of blood glucose excursions between meals including snacking, stress, illness, and medication compliance.”

It is noted that recitations herein of a component of the present invention being “configured” to embody a particular property or function in a particular manner, is a structural recitation, as opposed to a recitation of intended use. More specifically, the references herein to the manner in which a processor is “configured” denotes an existing physical condition of the processor and, as such, is to be taken as a definite recitation of the structural characteristics of the processor.

It is noted that terms like “preferably,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

It should now be understood that the systems and methods described herein may provide therapeutic guidelines to a person having diabetes. While particular embodiments and aspects of the present invention have been illustrated and described herein, various other changes and modifications may be made without departing from the spirit and scope of the invention. Moreover, although various inventive aspects have been described herein, such aspects need not be utilized in combination. It is therefore intended that the appended claims cover all such changes and modifications that are within the scope of this invention.

What is claimed is:

1. A method for providing therapeutic guidelines to a person having diabetes, the method comprising:
measuring a blood glucose (bG) level of the person for two or more days, wherein at least one bG measurement is taken per day, and the at least one daily bG measurement corresponds to one or more daily events for the person;
recording the measured bG levels in a computing device;
determining, by the computing device, whether the recorded bG levels are below, within, or above one or more predetermined bG ranges; and
automatically providing, by the computing device, therapeutic guidelines to the person, based on whether the recorded bG levels are below, within, or above the one or more predetermined bG ranges.

2. The method of claim 1, wherein the measuring of the bG level of the person takes place for three or more consecutive days.

3. The method of claim 1, wherein the one or more daily events for the person comprise eating breakfast, eating lunch, eating dinner, and going to sleep, such that at least seven bG measurements are taken per day: one before and after the person eats breakfast, lunch, and dinner, and one before the person goes to sleep.

4. The method of claim 3, wherein the bG measurements taken after the person eats breakfast, lunch, and dinner are taken approximately two hours after the person eats breakfast, lunch, and dinner.

5. The method of claim 4, wherein the one or more predetermined bG ranges comprise: a first predetermined bG range of approximately 81 milligrams of glucose per deciliter of blood (mg/dl) to approximately 110 mg/dl for bG measurements taken before the person eats breakfast, lunch, and dinner; and a second predetermined bG range of approximately 81 mg/dl to approximately 140 mg/dl for measurements taken after the person eats breakfast, lunch, and dinner and before the person goes to sleep.

6. The method of claim 1 further comprising determining whether the recorded bG levels are below or above the one or more predetermined bG ranges by at least a bG excursion amount, wherein the determining is performed by the computing device, and wherein automatically providing therapeutic guidelines to the person is further based on whether the recorded bG levels are below or above the one or more predetermined bG ranges by at least the bG excursion amount.

7. The method of claim 6, wherein the bG excursion amount is approximately 50 milligrams of glucose per deciliter of blood.

8. A computer-readable medium having computer-executable instructions for performing a method for providing therapeutic guidelines to a person having diabetes, the method comprising:
receiving measured blood glucose (bG) levels of the person into a computing device, wherein the measured bG levels of the person are taken for two or more days such that at least one bG measurement is taken per day, and the at least one daily bG measurement corresponds to one or more daily events for the person;
recording the measured bG levels in the computing device;
determining, by the computing device, whether the recorded bG levels are below, within, or above one or more predetermined bG ranges; and
automatically providing, by the computing device, therapeutic guidelines to the person, based on whether the recorded bG levels are below, within, or above the one or more predetermined bG ranges.

9. The computer-readable medium of claim 8, wherein the bG levels of the person are measured for three or more consecutive days.

10. The computer-readable medium of claim 8, wherein the one or more daily events for the person comprise eating breakfast, eating lunch, eating dinner, and going to sleep, such that at least seven bG measurements are taken per day: one before and after the person eats breakfast, lunch, and dinner, and one before the person goes to sleep.

11. The computer-readable medium of claim 10, wherein the bG levels of the person measured after the person eats breakfast, lunch, and dinner are measured approximately two hours after the person eats breakfast, lunch, and dinner.

12. The computer-readable medium of claim 11, wherein the one or more predetermined bG ranges comprise: a first predetermined bG range of approximately 81 milligrams of glucose per deciliter of blood (mg/dl) to approximately 110 mg/dl for measured bG levels taken before the person eats breakfast, lunch, and dinner; and a second predetermined bG range of approximately 81 mg/dl to approximately 140 mg/dl for measured bG levels taken after the person eats breakfast, lunch, and dinner and before the person goes to sleep.

13. The computer-readable medium of claim 8 further comprising determining whether the recorded bG levels are below or above the one or more predetermined bG ranges by at least a bG excursion amount, wherein the determining is performed by the computing device, and wherein automatically providing therapeutic guidelines to the person is further based on whether the recorded bG levels are below or above the one or more predetermined bG ranges by at least the bG excursion amount.

14. The computer-readable medium of claim 13, wherein the bG excursion amount is approximately 50 milligrams of glucose per deciliter of blood.

15. The computer-readable medium of claim 8, wherein the computer-readable medium comprises a compact disc (CD), a USB thumb drive, an optical drive, or a magnetic drive.

16. A blood glucose meter for providing therapeutic guidelines to a person having diabetes, the blood glucose meter comprising a processor, a memory, a display readable by the person, and a measuring element, wherein:
the measuring element is configured to measure the blood glucose (bG) level of the person for two or more days, wherein at least one bG measurement is taken per day, and the at least one daily bG measurement corresponds to one or more daily events for the person;
the processor is in electrical communication with the measuring element such that the processor is configured to read the bG level of the person measured by the measuring element;
the processor is in electrical communication with the memory such that the processor is configured to record the measured bG levels in the memory;
the memory comprises one or more predetermined bG ranges, such that the processor is configured to read the one or more predetermined bG ranges and the recorded bG levels and determine whether the recorded bG levels are below, within, or above the one or more predetermined bG ranges; and
the processor is in electrical communication with the display such that the processor is configured to transmit the therapeutic guidelines to the display, wherein the therapeutic guidelines are based on whether the recorded bG levels are below, within, or above the one or more predetermined bG ranges.

17. The blood glucose meter of claim 16, wherein measuring the bG levels of the person takes place for three or more consecutive days.

18. The blood glucose meter of claim 16, wherein the one or more daily events for the person comprise eating breakfast, eating lunch, eating dinner, and going to sleep, such that at least seven bG measurements are taken per day: one before and after the person eats breakfast, lunch, and dinner, and one before the person goes to sleep.

19. The blood glucose meter of claim 18, wherein the bG measurements taken after the person eats breakfast, lunch, and dinner are taken approximately two hours after the person eats breakfast, lunch, and dinner.

20. The blood glucose meter of claim 19, wherein the one or more predetermined bG ranges comprise: a first predetermined bG range of approximately 81 milligrams of glucose per deciliter of blood (mg/dl) to approximately 110 mg/dl for bG measurements taken before the person eats breakfast, lunch, and dinner; and a second predetermined bG range of approximately 81 mg/dl to approximately 140 mg/dl for measurements taken after the person eats breakfast, lunch, and dinner and before the person goes to sleep.

21. The blood glucose meter of claim 16 further comprising determining whether the recorded bG levels are below or above the one or more predetermined bG ranges by at least a bG excursion amount, and providing the therapeutic guidelines to the person is further based on whether the recorded bG levels are below or above the one or more predetermined bG ranges by at least the bG excursion amount.

22. The blood glucose meter of claim 21, wherein the bG excursion amount is approximately 50 milligrams of glucose per deciliter of blood.
